(19) United States
(12) Patent Application Publication
(10) Pub. No.: US 2019/0046783 A1
(43) Pub. Date: Feb. 14, 2019

(54) SYSTEMS AND METHODS FOR STERILE CATHETER CONNECTION

(71) Applicant: The Regents of the University of California, Oakland, CA (US)

(72) Inventors: Shyam Raghavan, Oakland, CA (US); Hanmin Lee, Oakland, CA (US); Dillon Kwiat, Oakland, CA (US); Neil Ray, Oakland, CA (US)

(21) Appl. No.: 15/759,917

(22) PCT Filed: Sep. 15, 2016
(86) PCT No.: PCT/US16/51941

§ 371 Date: Mar. 14, 2018

Related U.S. Application Data
(60) Provisional application No. 62/219,109, filed on Sep. 15, 2015.
(60) Provisional application No. 62/315,243, filed on Mar. 30, 2016.

(51) Int. Cl. A61M 39/18; A61M 39/10; A61M 39/26

(52) U.S. Cl. CPC A61M 39/18; A61M 39/1011; A61M 2039/1066; A61M 2039/1027; A61M 39/26

(57) ABSTRACT

In one embodiment, a sterile connector includes an inner member having a connection fitting configured to couple with a separate component that is to be connected to the sterile connector and an inner passage through which fluids can travel, and an outer member in which the inner member is received, the outer member having an inner sealing mechanism that seals the connection fitting within the outer member to protect the connection fitting from contamination, wherein the connection fitting can be passed through the sealing mechanism and extended from the outer member to enable the connection fitting to couple with the separate component.

### CROSS-REFERENCE TO RELATED APPLICATIONS

This application claims priority to co-pending U.S. Provisional Application Ser. No. 62/219,109, filed Sep. 15, 2015, and U.S. Provisional Application Ser. No. 62/315,243, filed Mar. 30, 2016, both of which are hereby incorporated by reference herein in their entireties.

### BACKGROUND

Central venous catheters are often used to deliver liquids (e.g., medications) to a vein of a patient. Such catheters typically have one or more ports through which the liquids can be delivered to a main lumen of the catheter for delivery into the vein. Unfortunately, these ports are exposed to their surroundings and are susceptible to contamination that can spread to the main lumen of the catheter and, ultimately, to the patient. It would be desirable to have a system and method for protecting the catheter lumens so that they are not exposed prior, during, and/or after use to their surroundings.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a side view of a first embodiment of a sterile connection system.

FIG. 2A-2D are sequential side views illustrating operation of the system shown in FIG. 1.

FIG. 3 is a side view of a second embodiment of a sterile connection system.

FIG. 4 is a distal perspective view of a catheter connector of the system of FIG. 3.

FIG. 5 is a proximal perspective view of the catheter connector of FIG. 3.

FIG. 6 is an exploded perspective view of the catheter connector of FIGS. 4 and 5.

FIG. 7 is a proximal perspective view of an outer member of the catheter connector of FIGS. 4-6.

FIG. 8 is a cross-sectional perspective view of an inner member of the catheter connector of FIGS. 4-6.

FIG. 9 is a distal perspective view of a distal portion of the inner member of

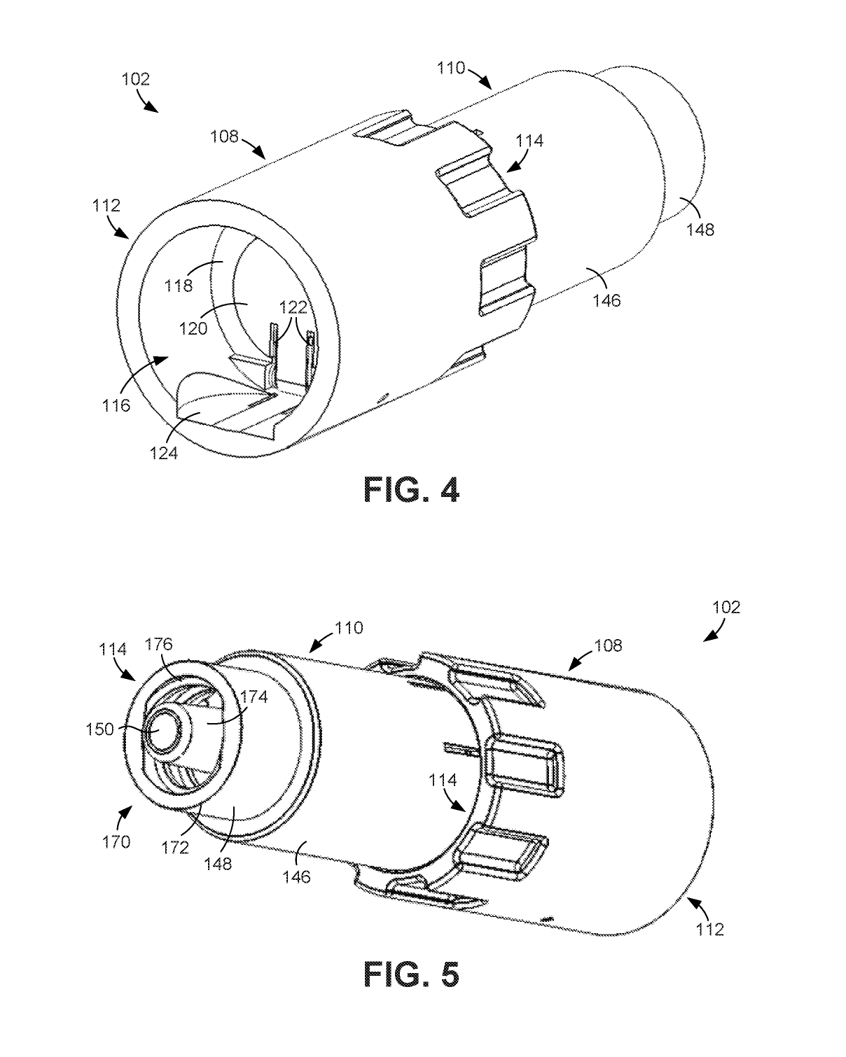

FIG. 8. FIG. 10 is a cross-sectional side view of the catheter connector of FIGS. 4-6.

FIGS. 11A-11C are sequential distal perspective views of the catheter connector of FIGS. 4-6 illustrating extension of a connection fitting from the connector.

### DETAILED DESCRIPTION

As described above, it would be desirable to have a system and method for protecting the lumens of a catheter, such as a central venous catheter, so that they are not exposed to their surroundings prior, during, and/or after administration of a liquid to the patient using the catheter. Examples of such systems and methods are described herein. In some embodiments, a connection system comprises a catheter connector that is configured to releasably connect to another component, such as an infusion line. When not in use, the portion of the catheter connector that interfaces with the other component is protected from contamination by an inner sealing mechanism. When the catheter connector is to be connected to another component, however, the sealing mechanism is opened to facilitate this connection.

In the following disclosure, various specific embodiments are described. It is to be understood that those embodiments are example implementations of the disclosed inventions and that alternative embodiments are possible. All such embodiments are intended to fall within the scope of this disclosure.

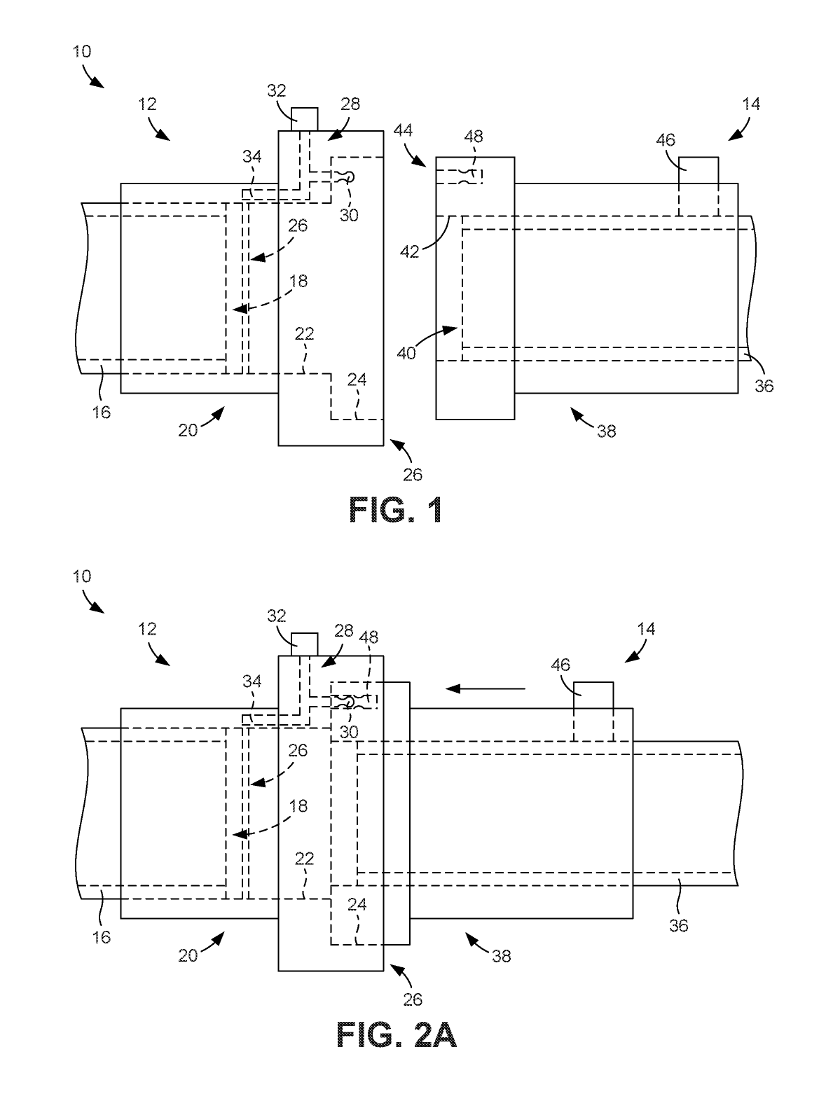

FIG. 1 illustrates a first embodiment of a connection system 10 that can be used to connect a catheter 12, such as a central venous catheter, to another component, such as an infusion line 14. Although the central venous catheter application has been explicitly identified, it is noted that other applications are possible. For example, the connection system 10 could alternatively be used to form connections for tube extenders, medical three-way stopcocks, arterial line set-ups, or drug allocation.

As shown in FIG. 1, the catheter 12 includes a lumen 16, such as a flexible tube, through which fluid can flow. In embodiments in which the catheter 12 is a central venous catheter, the lumen 16 can comprise a secondary lumen that feeds into a primary lumen that is inserted into the patient's vein. Mounted to a free end 18 of the lumen 16 is a connector 20 that can be used to connect the infusion line 14 to the catheter 12. As indicated in FIG. 1, the connector 20 includes a first inner opening 22 in which the lumen 16 is received. In addition, the connector 20 includes a larger second inner opening 24 near its free end 18 that is configured to receive a connector of the infusion line 14.

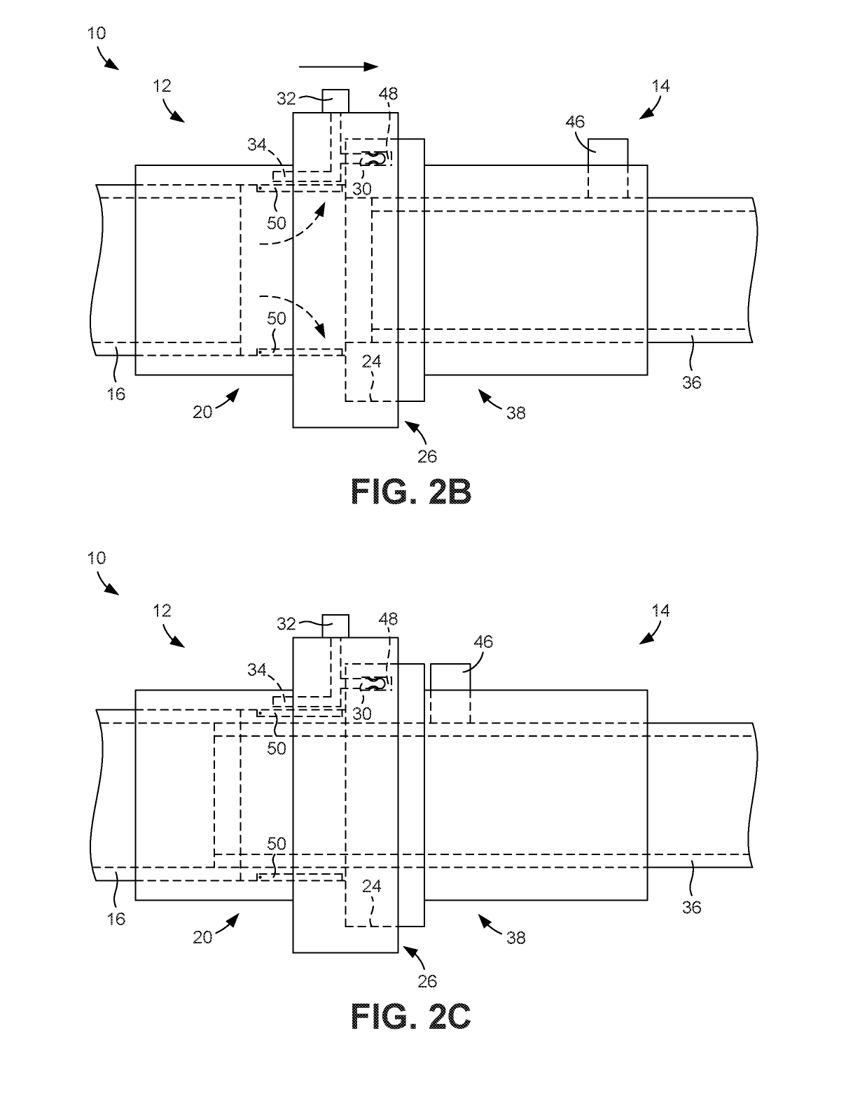

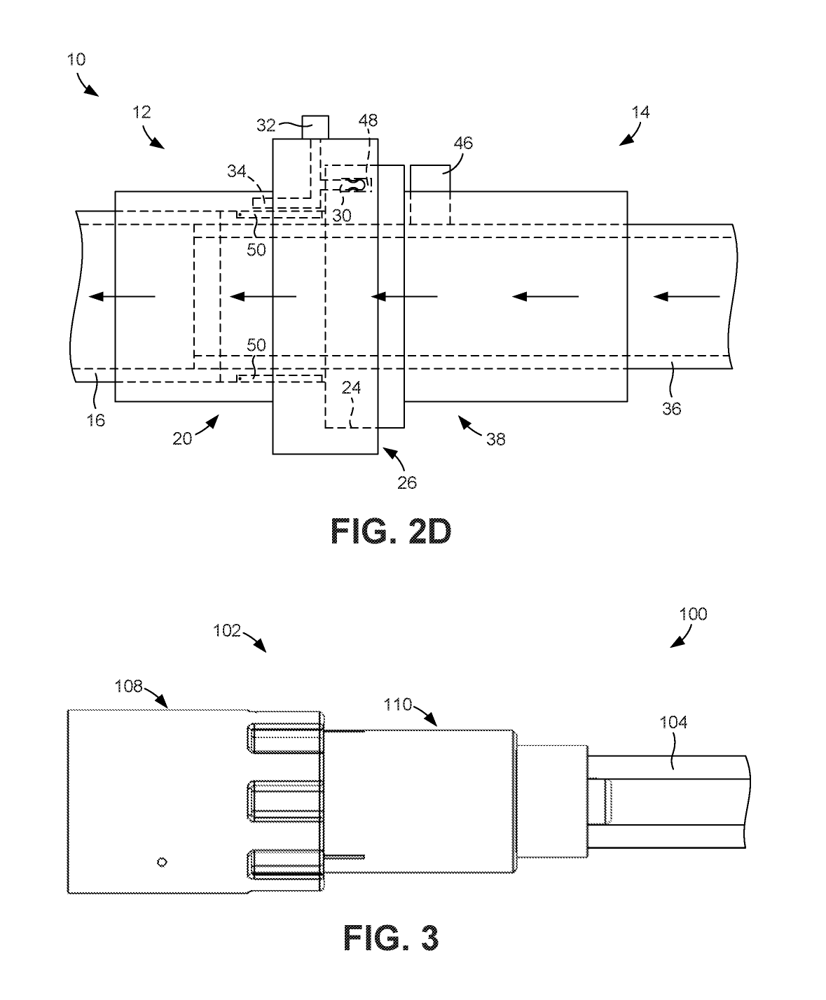

Provided within the first inner opening 22 is an inner sealing mechanism 26 that seals the free end 18 and interior of the lumen 16 from the outside environment. The sealing mechanism 26 is configured to remain in a closed orientation illustrated in FIG. 1 until the connector 20 is locked with the connector of the infusion line 14. When such locking has been achieved, however, the sealing mechanism 26 can be transitioned to an open orientation (see, e.g., FIG. 2B) in which a lumen of the infusion line 14 can pass through the sealing mechanism and connect to the lumen 16. In some embodiments, the sealing mechanism 26 comprises a gate with opposed doors that can open and close. In other embodiments, the sealing mechanism 26 comprises an iris diaphragm that can open and close using a rotary action.

The connector 20 further comprises a locking mechanism 28 that can be used to lock the connector to the connector of the infusion line 14. In some embodiments, the locking mechanism 28 includes a locking element 30 that can be extended and retracted to alternately lock and unlock the connectors. Such extension and retraction can be effected, in some embodiments, using an external actuator 32, such as a slide. In addition, the locking mechanism 28 can include a sealing mechanism locking element 34 that enables the sealing mechanism to transition from the closed orientation to the open orientation once the connectors have been locked together.

Like the catheter 12, the infusion line 14 includes a lumen 36, such as a flexible tube, through which fluid can flow, and a connector 38 that is mounted to a free end 40 of the lumen. More specifically, the lumen 36 is received within an inner opening 42 of the connector 38. In this case, however, the lumen 36 can axially move relative to the inner opening 42 and, therefore, the connector 38. In particular, the lumen 36 can be alternately extended from a free end 44 of the connector 38 and retracted back into the connector using an extension mechanism. In some embodiments, the extension mechanism comprises an external actuator 46, such as a slide.

With further reference to FIG. 1, the connector 38 of the infusion line 14 includes a locking opening 48 that is configured to receive the locking element 30 of the locking mechanism 28 to facilitate locking of the connector 20 to the connector 38.

FIGS. 2A-2D are sequential drawings that illustrate sterile connection of the infusion line 14 to the catheter 12 and the delivery of fluid from the infusion line to the catheter. Beginning with FIG. 2A, the infusion line 14 can be connected to the catheter 12 by inserting the free end 44 of the infusion line connector 38 into the second inner opening 24 of the catheter connector 20. As shown in FIG. 2A, the locking element 30 of the locking mechanism 28 of the catheter connector 20 can enter the locking opening 48 of the infusion line connector 38 to ensure correct alignment of the two connectors.

Once the infusion line connector 38 has been received by the catheter connector 20 in the manner illustrated in FIG. 2A, the two connectors can be locked together using the locking mechanism 28. FIG. 2B illustrates such locking. In particular, the external actuator 32 provided on the catheter connector 20 has been slid toward the infusion line connector 38 so as to drive the locking element 30 deeper into the locking opening 48. Once the locking element 30 has been fully inserted into the opening 48, the two connectors 20, 38 are securely locked to each other and cannot be separated from each other until the locking mechanism is returned to its original unlocked orientation.

In addition to locking the connectors 20 and 38 together, the actuation of the locking mechanism 28 releases the sealing mechanism 26 to enable it to open and provide access to the catheter lumen 16. In some embodiments, the actuation of the locking mechanism 28 moves the sealing mechanism locking element 34 out of the way so that it no longer prevents such opening of the sealing mechanism 26. In the example of FIG. 2B, the sealing mechanism 26 is assumed to comprise a gate having opposed doors 50 that swing open to provide access to the lumen 16. In some embodiments, the doors 50 can automatically open once the connectors 20, 38 are locked under a spring force. In other embodiments, the doors 50 can be manually opened by a user by manipulating an external actuator, such as a further slide (not shown).

At this point, the infusion line 14 is locked onto the catheter 12 and the sealing mechanism 26 has been opened. Next, the infusion line lumen 36 can be connected to the catheter lumen 16 to enable fluid to flow from the infusion line lumen and into the catheter lumen. In some embodiments, this connection can be achieved using the external actuator 46. In particular, the actuator 46 can be slid toward the catheter connector 20 so as to extend the infusion line lumen 36 from the connector, through the open sealing mechanism 26 (e.g., doors 50), and into the catheter lumen 16, as illustrated in FIG. 2C. When this has been achieved, the two lumens 16, 36 are connected and form a continuous path for fluid to flow, as illustrated by the flow arrows in FIG. 2D.

FIGS. 3-11 illustrate a second embodiment of a connection system 100 that can be used to connect a catheter, such as a central venous catheter, to another component, such as an infusion line. As with the first embodiment, the connection system 100 includes a sterile catheter connector 102 to which the other component can be releasably connected. FIG. 3 shows the connector 102 mounted to a lumen 104 of a catheter, such as a secondary lumen of a central venous catheter.

FIGS. 4 and 5 illustrate the catheter connector 102 in perspective views independent of the catheter on which it is normally mounted. As shown in these figures, the connector 102 generally comprises a distal, outer member 108 and a proximal, inner member 110 that is partially housed within the outer member. As described in greater detail below, these two members 108, 110 can be rotated relative to each other along their concentric longitudinal axes to extend or retract a connection fitting of the connector to facilitate its releasable connection to another component.

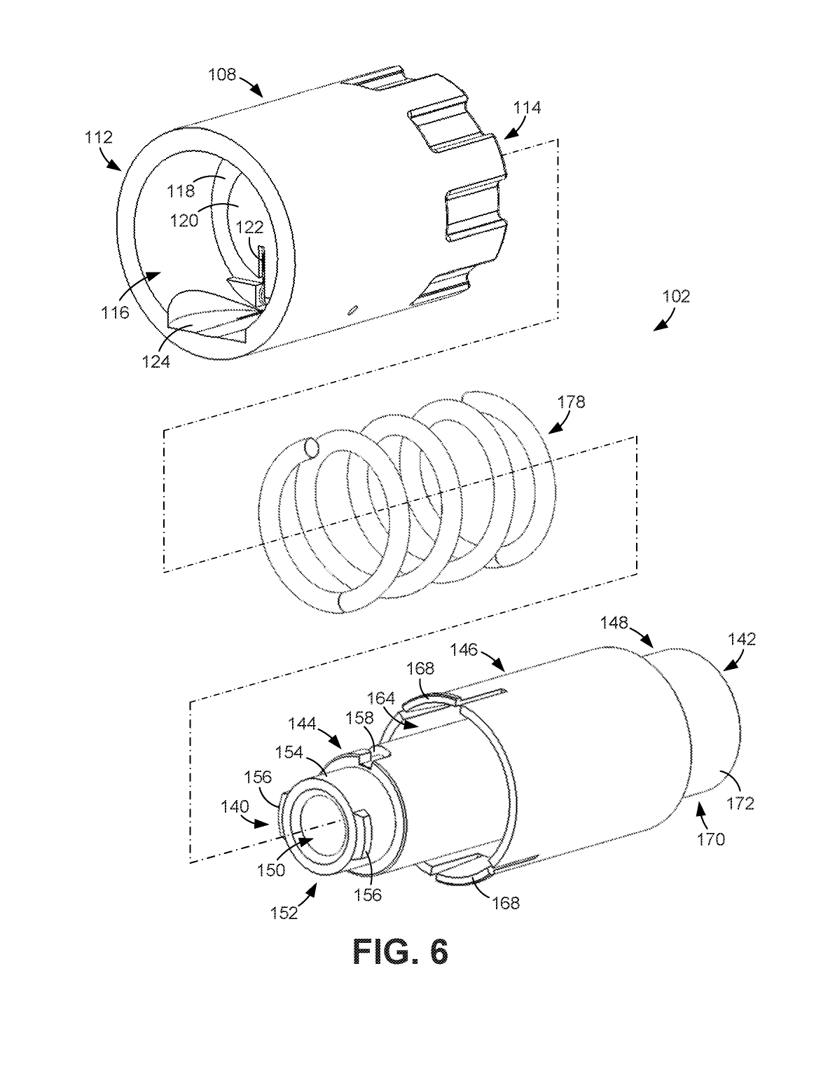

FIG. 6 shows the catheter connector 102 in an exploded perspective view in which the distal, outer member 108 is separated from the proximal, inner member 110. As is apparent in FIG. 6, the outer member 108 is generally cylindrical and includes a distal end 112 and a proximal end 114. Provided at the distal end 112 of the outer member 108 is a cylindrical distal cavity 116 that extends from the distal end to a transverse inner wall 118. An opening (not visible in FIG. 4) if formed through the wall 118 through which a connection fitting of the proximal, inner member 110 can pass when another component is to be connected to the catheter connector 102. Protecting this fitting from contamination when it is not needed for connection purposes is an inner sealing mechanism that comprises a pivotable gate 120 that seals the opening when the gate is in the closed position shown in FIG. 6. In some embodiments, the gate 120 is biased toward the closed position by one or more biasing elements, such as torsion springs 122. As is further shown in FIG. 6, the distal cavity 116 can include a void 124 formed in the wall of the outer member 108 that provides space for the gate 120 when it is in an open position (see FIG. 11C).

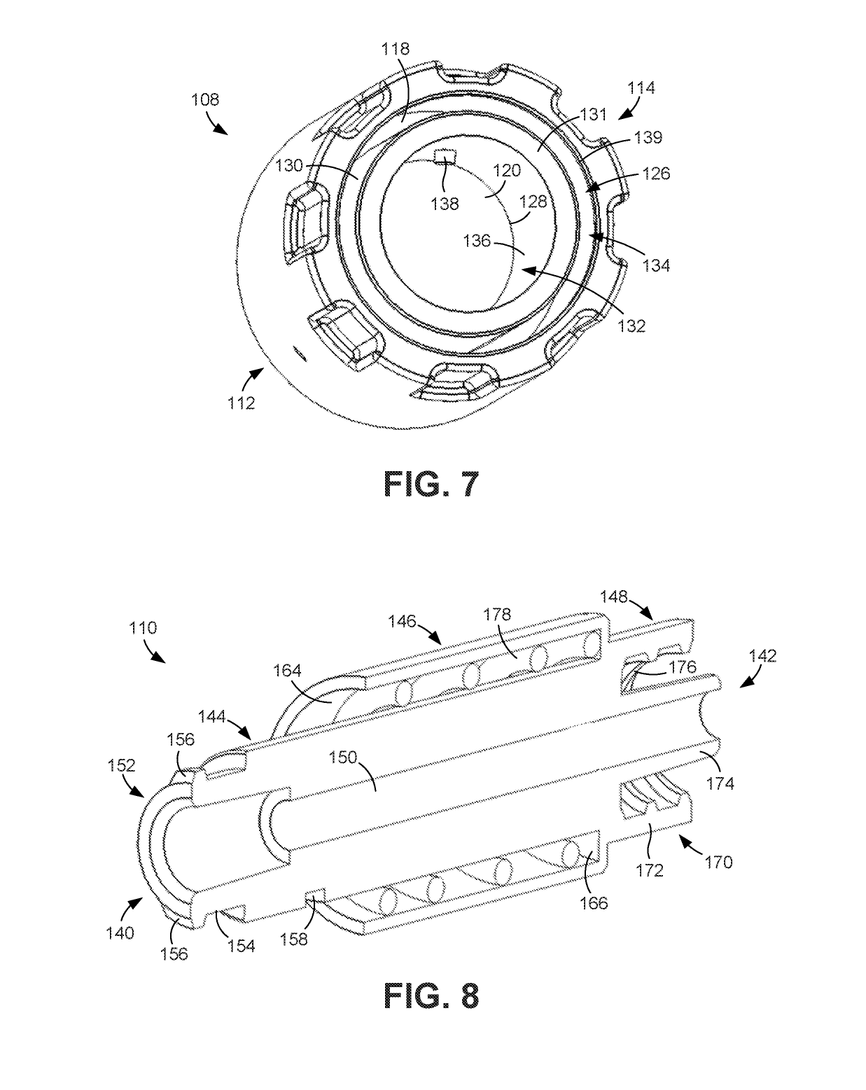

The proximal end 114 of the distal, outer member 108 is shown in FIG. 7. As depicted in this figure, the outer member 108 further comprises a cylindrical proximal cavity 126 that is configured to receive the proximal, inner member 110. The proximal cavity 126 extends from the proximal end 114 of the outer member 108 to the inner wall 118. The aforementioned opening 128 in this wall 118 is visible in FIG. 7, as is the gate 120 that seals it closed. Provided within the proximal cavity 126 is a concentric inner tube 130 that extends from the wall 118 to the proximal end 114 of the outer member 108 to form a planar end face 131. As such, the inner tube 130 divides the proximal cavity 126 into an inner cylindrical portion 132 and an outer cylindrical portion 134. Provided on an inner surface 136 of the inner tube 130 is a pin 138 that is configured to follow a slot formed in the inner member 110. Provided at the proximal end 114 of the outer member 108 is an inner lip 139 that extends inward into the outer cylindrical portion 134 of the cavity 126.

With reference back to FIG. 6, the proximal, inner member 110 is also generally cylindrical and includes a distal end 140 and a proximal end 142. The inner member 110 can be described as comprising a distal portion 144, a medial portion 146, and a proximal portion 148, each of which can be unitarily formed together from the same piece of material. The nature of these portions of the inner member 110 is most clearly visible in the cross-sectional view of FIG. 8.

As indicated in FIG. 8, the distal portion 144 is an elongated tubular element that extends from the medial portion 146. The distal portion 144 is configured to be received within the inner tube 130 of the distal, outer member 108 and defines part of an inner fluid passage 150 through which fluids to be delivered to the catheter can travel (i.e., from the distal end 140 to the proximal end 142). Formed at the distal end of the distal portion 144 is a connection fitting 152 of the catheter connector 102. As shown in FIG. 8, this fitting 152 can take the form of a luer-lock fitting that includes a relatively narrow neck 154 and two laterally extending flanges or tabs 156 that are configured to be received by an internally threaded connector of the other component that is to be connected to the catheter connector 102 (e.g., infusion line).

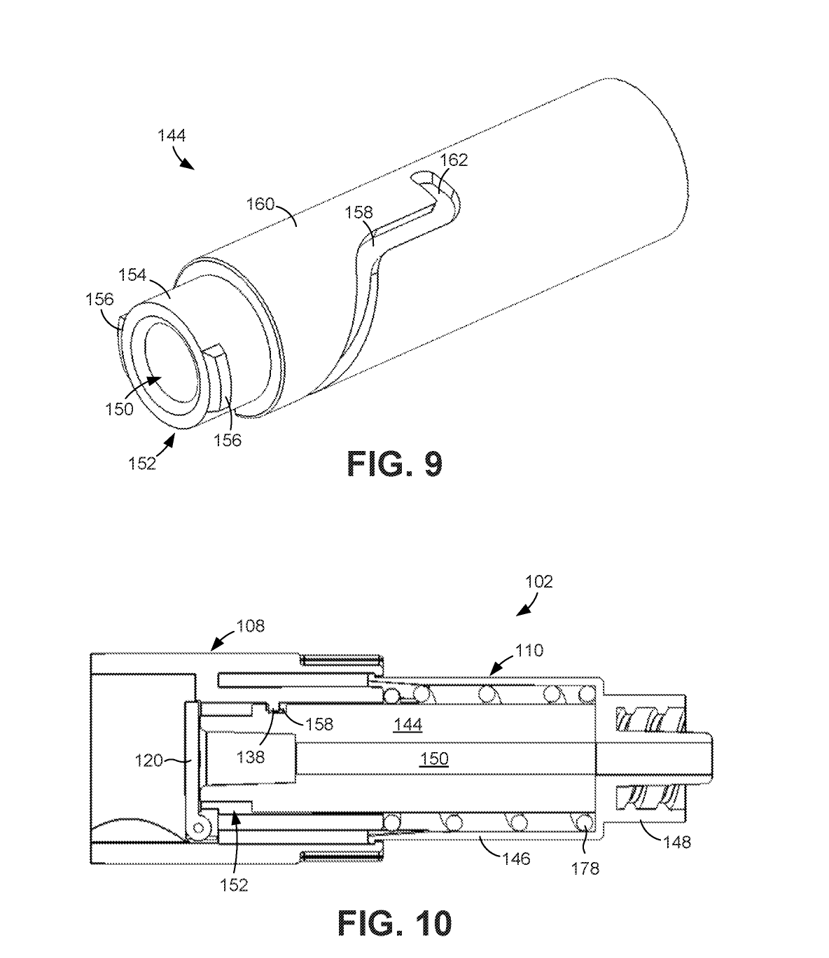

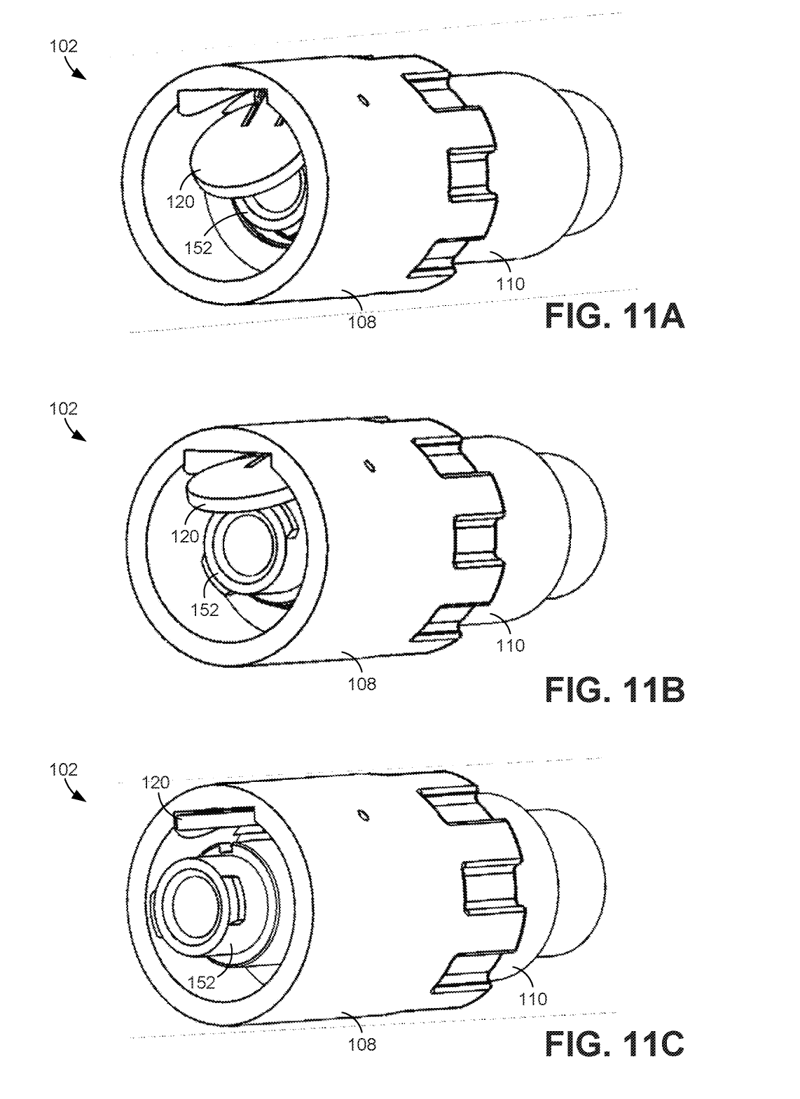

With reference back to FIG. 6, the distal portion 144 of the proximal, inner member 110 further includes an elongated slot 158 formed in an outer surface 160 of the portion that is configured to receive the pin 138 of the distal, outer member 108. This slot 158 is also illustrated in FIG. 9, which shows the distal portion 144 independent of the remainder of the inner member 110. As is depicted in FIG. 9, the slot 158 follows a curved, diagonal path along part of the length of the distal portion 144. The slot 158 terminates at a proximal-most location with a lateral portion 162 that enables the catheter connector 102 to be locked in an orientation in which the connection fitting 152 extends out from the outer member 108 and, therefore, is prepared for connection to another component.

With reference again to FIG. 8, the medial portion 146 of the proximal, inner member 110 is a hollow cylindrical element that is configured to pass over the inner tube 130 of the distal, outer member 108. The medial portion 146 defines a cylindrical cavity 164 that extends from the distal end of the portion to an inner wall 166. The distal portion 144 extends distally from this wall 166 and, therefore, much of the distal portion's length is contained within the cavity 164, as shown in FIG. 8. With reference again to FIG. 6, the medial portion 146 also includes outwardly biased tabs 168 at its distal end that are configured to engage the inner lip 139 of the distal, outer member 108 to prevent the inner member 110 from withdrawing and decoupling from the outer member.

Referring once again to FIG. 8, the proximal portion 148 of the proximal, inner member 110 forms a further connection fitting 170 that is configured to mount the connector 102 to a lumen of a catheter, such as a secondary lumen of a central venous catheter. As illustrated in FIG. 8, the fitting 170 includes an outer tube 172 that partially surrounds an inner tube 174. The outer tube 172 comprises internal threads 176 that can receive a lumen.

With reference back to FIG. 6, the catheter connector 102 further includes a compression spring 178 that is interposed between the distal, outer member 108 and the proximal, inner member 110. As shown in FIG. 8, this spring 178 is configured to be positioned within the cylindrical cavity 164 that exists between the distal portion 144 and the medial portion 146 of the inner member 110. When the connector 102 is assembled, the distal end of this spring 178 abuts the end face 131 of the inner tube 130 of the outer member 108 (see FIG. 10). Positioned in this manner, the spring 178 urges the outer and inner members 108, 110 in opposite directions so as to bias the connector 102 toward a fully retracted orientation in which the connection fitting 152 is contained and sealed within the connector.

FIG. 10 shows the assembled catheter connector 102 in side cross-section. In this figure, the connector 102 is in the fully retracted orientation in which the connection fitting 152 is contained and sealed within the connector. As can be appreciated from this figure, the fitting 152 is positioned within the inner tube 130 of the distal, outer member 108 adjacent to its gate 120, which is pressed against the inner wall 118 under the force of the torsion springs 122. As can also be appreciated from this figure, the pin 138 of the inner tube 130 of the outer member 108 is received within the slot 158 of the distal portion of the inner member 110. Because of the presence of the pin 138 within the slot 158, counter-clockwise rotation of the outer member 108 relative to the inner member 110 causes the outer member to move linearly in a proximal direction relative to the inner member against the force of the compression spring 178. This relative linear movement causes the fitting 152 to push open the gate 120 against the force of the torsion springs 122. In this manner, continued twisting of the outer member 108 causes the connector 102 to compress and the fitting 152 to emerge from the distal end 114 of the outer member 108. This process is illustrated in the sequential views of FIGS. 11A-11C.

As can be appreciated from FIG. 11C, which shows a fully extended orientation of the catheter connector 102, once the outer member 108 has been rotated to the maximum extent relative to the inner member 110, the connection fitting 152 extends beyond the distal end 112 of the connector 102 so that it is available for connecting with a mating connector of another component, such as an infusion line. Notably, the connector 102 can be locked in the fully extended orientation shown in FIG. 11C by positioning the outer member 108 such that its pin 138 is positioned within the lateral portion 162 of the slot 158.

If desired, the catheter connector 102 can be returned to the fully retracted orientation shown in FIG. 10. In particular, the distal, outer member 108 can be rotated in the clockwise direction relative to the proximal, inner member 110. Once the pin 138 leaves the lateral portion 162 of the slot 158, the force of the compression spring 178 will automatically return the outer member 108 to its original position. In similar manner, the torsion springs 122 will return the gate 120 to its original closed position.

Claimed are:

1. A sterile connector comprising:
an inner member including a connection fitting configured to couple with a separate component that is to be connected to the sterile connector and an inner passage through which fluids can travel; and
an outer member in which the inner member is received, the outer member including an inner sealing mechanism that seals the connection fitting within the outer member to protect the connection fitting from contamination, wherein the connection fitting can be passed through the sealing mechanism and extended from the outer member to enable the connection fitting to couple with the separate component.

2. The connector of claim 1, wherein the sealing mechanism comprises a gate that can be pivoted from a closed position to an open position.

3. The connector of claim 2, wherein the outer member further includes a inner wall having an opening through which the connection fitting can pass and wherein the gate seals the opening in the closed position.

4. The connector of claim 3, wherein the gate is biased toward the closed position with a spring.

5. The connector of claim 2, wherein the outer member can be rotated relative to the inner member and wherein such rotation causes the connection fitting to push the gate open.

6. The connector of claim 5, wherein the inner member includes a curved slot that extends along part of its length and the outer member includes a pin that is received within the slot, wherein the pin travels along the slot when the outer member is rotated relative to the inner member.

7. The connector of claim 6, wherein the inner member comprises a distal portion and the connection fitting is provided on the distal portion.

8. The connector of claim 7, wherein the slot is formed in an outer surface of the distal portion.

9. The connector of claim 8, wherein the outer member further includes a proximal cavity and an inner tube that separates the proximal cavity into an inner portion and an outer portion, wherein the distal portion of the inner member is received within the inner portion of the proximal cavity and the pin is provided on an inner side of the inner tube.

10. The connector of claim 9, further comprising a spring positioned between the inner and outer members that opposes rotation of the outer member relative to the inner member.

11. The connector of claim 10, wherein the inner member further includes a medial portion that is received within the outer portion of the proximal cavity of the outer member, the medial portion surrounding part of the distal portion so as to define a cylindrical cavity between the medial and distal portions, wherein the spring is positioned within the cylindrical cavity.

12. The connector of claim 11, wherein the medial portion includes an inner wall and wherein the spring contacts the inner wall at one end and an end face of the inner tube of the outer member at its other end.

13. The connector of claim 12, wherein the inner member further includes a proximal portion that comprises a further connection fitting configured to mount the connector to a lumen.

14. The connector of claim 13, wherein the further connection fitting comprises internal threads.

15. The connector of claim 1, wherein the connection fitting comprises a luer-lock fitting that includes a neck and laterally extending tabs that extend from the neck.

16. A sterile connector comprising:
an internal sealing mechanism configured to protect a lumen to which the connector is mounted from being contaminated, the sealing mechanism comprising a gate that can be closed to seal the connector and opened to enable fluids to pass through the connector.

17. A method for preventing contamination of a catheter connector, the method comprising:
containing a connection fitting within the connector behind a sealing mechanism; and
extending the connection fitting through the sealing mechanism when the connecting fitting is to be coupled with a separate component.

18. The method of claim 17, wherein the sealing mechanism comprises a gate that can be pivoted from a closed position to an open position.

19. The method of claim 18, wherein the connector comprises an outer and inner members and wherein rotation of the outer member relative to the inner member causes the connection fitting to push the gate open.

20. The method of claim 19, wherein the gate is biased toward the closed position with a spring.
